(19) United States
(12) Patent Application Publication
(10) Pub. No.: US 2006/0276455 A1
(43) Pub. Date: Dec. 7, 2006

(54) Use of a mast cell activation or degranulation blocking agent in the manufacture of a medicament for the treatment of cerebral ischemia

(76) Inventors: Perttu J. Lindsberg, Helsinki (FI); Marja-Liisa Karjalainen-Lindsberg, Helsinki (FI); Turgut Tatlisumak, Helsinki (FI); Daniel Strbian, Helsinki (FI)

Correspondence Address: SUGHRUE MION, PLLC, 2100 PENNSYLVANIA AVENUE, N.W., SUITE 800, WASHINGTON, DC, 20037, US

(21) Appl. No.: 10/545,324

(22) PCT Filed: Feb. 13, 2004
(86) PCT No.: PCT/FI04/00071

§ 371 Date: Jun. 16, 2006

Related U.S. Application Data
(60) Provisional application No. 60/447,003, filed on Feb. 13, 2003.

(51) Int. Cl. A61K 31/55; A61K 31/495; A61K 31/4172; A61K 31/353

(52) U.S. Cl. 514/217.05; 514/255.04; 514/456; 514/400

(57) ABSTRACT

The invention concerns the use of a mast cell activation- or degranulation-blocking agent in the manufacture of a medicament for the treatment of cerebral ischemia. The invention also relates to treatment of patients suffering from acute ischemic stroke, acute hemorrhagic stroke, subarachnoid hemorrhage, cerebral venous thrombosis or global cerebral ischemia associated with cardiac arrest. Further, the invention provides compositions of contrast media or similar exogenous media, which are intended for use in diagnostic or therapeutic applications for introduction into the intravascular, intrathecal or the intracranial space, comprising a mast cell degranulation-blocking and/or mast cell activation-blocking agent.

### BACKGROUND OF THE INVENTION

1. Field of the Invention

The present invention relates to new therapeutic uses of known compounds. In particular, the invention relates to the use of mast cell activating- and degranulation-blocking agents for preventing and treating cerebral ischemia, which otherwise may cause ischemic brain edema.

2.Description of Related Art

There are life-threatening conditions of the central nervous system, where the volume of the brain substance enlarges or there is an extracerebral rapid expansion of the intracranial compartment encapsulated by the meninges. These conditions include all major hemorrhagic strokes and intracranial hematomas. There are only limited cerebral reserve spaces in the intracranial space, which can shrink to accommodate the above increases in the volumes of the intracranial compartments. The venous and cerebrospinal fluid compartments can, to a modest extent, shrink. Sometimes, as in cerebral ischemia and hemorrhage, the brain matter itself undergoes ischemic edematous change. It is a common clinical situation, that a patient needs to be rushed to the neurosurgical therapy to prevent increase of intracranial pressure and brain herniation, a downward volumetric shift of the brain matter that compresses the brain stem often killing the patient. Even in a milder form, edema can cause secondary brain matter through microvascular failure.

At present, there are no specific therapy forms for preventing cerebral edema. Already formed brain edema is treated by intravenous infusion of hyperosmolalic solutions (e.g. mannitol, glycerol, hypertonic solutions, and starch), which passively and non-specifically withdraw fluid from the extracellular space. They are associated with untoward hemodynamic and metabolic alterations. Barbiturate anesthesia decreases the intracranial pressure, but often compromises hemodynamic state and requires prolonged artificial respirator treatment and its associated complications. Hyperventilation is used to decrease blood carbon dioxide level so to induce vasoconstriction, but its therapeutic time window is limited only to a few hours and can lead to tissue ischemia. Occasionally, surgical decompressive operations are performed on the cranium, to allow brain matter to enlarge without hazardous rise in intracranial pressure. Invasive procedures to treat brain edema are naturally costly and associated with unavoidable complications.

For the reasons, there is a great need for alternative or additional methods for preventing or treating brain edema, a volumetric expansion of brain matter or intracranial tissue.

### SUMMARY OF THE INVENTION

It is an object of the present invention to provide a new use of mast cell degranulation-blocking and/or mast cell activation-blocking agents.

It is another object of the present invention to provide a novel method for treating patients suffering from cerebral ischemia subjected to conditions conducive to cerebral ischemia.

It is a third object of the present invention to provide novel compositions for use as contrast media or other exogenous media in diagnostic or therapeutic applications for introduction into the intravascular, intrathecal or the intracranial space.

These and other objects, together with the advantages thereof over the known therapeutic uses, which shall become apparent from specification which follows, are accomplished by the invention as hereinafter described and claimed.

Mast cells are present in the brain and located typically in perivascular spaces. They contain substantial amounts of preformed pro-inflammatory, vasoactive, anticoagulant and proteolytic substances. These substances are contained within numerous intracytoplasmic granules. They can synthesise a large number of additional substances. Generally, they protect parenchymal organs from exogenous, hazardous agents such as microbes and toxic or allergenic particles. They can influence the permeability of small cerebral vessels, regulate blood circulation and prime immunological responses. They can also mount an immediate host defence response by rapid degranulation, which can lead to hazardous anaphylactic and other systemic bodily reactions. Mast cells have been found in most parenchymal organs, including the brain and meninges.

The present invention is based on the observation that mast cells present in the brain tissue, surrounding small cereral vessels in perivascular spaces, increase in number and commonly degranulate, during and following induced cerebral ischemia. The mast cells colocalize with perivascular edema formation and early leukocyte emigration from the blood circulation. It would therefore seem that the tissue-based, stationary mast cells already in place to participate in the initial response are in part causing later events leading to florid blood-brain barrier damage and the entry of inflammatory cells from the circulation, which can aggravate the brain injury.

In the connection with the present invention, we have found that mast cell degranulation participates to a significant degree in ischemic brain edema formation, and the vasogenic component is substantial in the early phase of brain edema development. Based on the above observations, the present new method of treating cerebral ischemia has been deviced, which method comprises administering a therapeutically effective amount of a mast cell activation and/or degranulation-blocking agent will reduce cerebral ischemia. More specifically, according to the invention, a mast cell degranulation-blocking and/or mast cell activation-blocking agent is used in the manufacture of a medicament for the treatment of cerebral ischemia in a patient.

The invention also comprises the use of a mast cell degranulation-blocking and/or mast cell activation-blocking agent in the manufacture of a medicament for preventing or reducing mast cell degranulation in a patient subjected to conditions conducive to cerebral ischemia.

Further, the invention comprises contrast media and similar exogenous media which are used in diagnostic or therapeutic applications for introduction into the intravascular, intrathecal or the intracranial space and which, according to the present invention, contain at least one mast cell degranulation-blocking and/or mast cell activation-blocking agent in a therapeutically effective amount to prevent or significantly reduce any degranulation or activation of the mast cells resulting from the use of the contrast medium or other exogenous medium.

In the following, the term “mast cell degranulation-blocking agent” will be used interchangeably with “mast cell degranulation-blocking and/or mast cell activation-blocking agent” to designate an agent having either or both of the two activities: mast cell degranulation blocking and mast cell activation blocking. Mast cell degranulation seems to be of particular importance for the formation of cerebral ischemia and ischemic edema, as will be explained below, although mast cell activation is also deemed to be harmful.

By using substances, such as cromoglycate, whose pharmacological effect consists essentially exclusively of affecting the degranulation or activation of mast cells or inhibiting the main granula constituents of cerebral mast cells, it is possible to treat cerebral ischemia. For the purpose of the present invention, cromoglycate and the similar compounds, such as any 2-carboxylatomchromon-5′-yl-2-hydroxypropane derivatives, nedocromil and tranilast, are considered to be agents, which specifically prevent or reduce mast cell degranulation or activation, in the sense that they do not have other major tissue activity except for that exhibited through their mast cell degranulation or activation blocking mechanism. Similarly, as will be discussed in more detail below, inhibitors of the c-kit receptors on mast cells as well as chymase and procollagenase activators can be used in the present invention.

Further, the invention comprises a method of treating a patient suffering from cerebral ischemia, comprising the step of administering to the patient a pharmaceutically effective amount of a mast cell degranulation-blocking agent, and another method of preventing or reducing mast cell degranulation in a patient subjected to conditions conducive to cerebral ischemia, comprising administering a therapeutically effective amount of a of a mast cell degranulation-blocking agent.

By the present invention, a significant reduction in ischemic edema of up to 40% compared to control can be obtained (this figure is based on the animal experiments described in detail below). The finding was surprising, since it has long been held that the immediate phase of brain edema is caused by cytotoxic mechanism leading to a shift of extracellular fluid, not intravascular fluid, into the cerebral cellular compartment.

The invention also provides efficient precaution against cerebral edematous change in conditions potentially conducive to cerebral ischemia caused by, e.g., vascular neurosurgery, heart surgery, carotid artery endarterectomies or surgery to other major arteries supplying the central nervous tissue; surgical procedures associated with physical brain handling of brain and meningeal tissues; radiation therapy or diagnosis, that could cause significant mast cell degranulation or activation through mechanical forces or through electromagnetic radiation; and treatments involving the administration of contrast media or other exogenous media, which may also degranulate or activate mast cells, for diagnostic or therapeutic purpose into the intravascular, intrathecal or the intracranial space.

Next, the invention will be examined more closely with the aid of a detailed description and some working examples.

### BRIEF DESCRIPTION OF THE DRAWINGS

FIG. 1 is a bar chart showing the percentual hemispheric expansion caused by focal cerebral ischemia in rats after pretreatment with two different substances in comparison to control;

FIG. 2 is a bar chart showing the fluorescence above autofluorescence threshold for the same samples as in FIG. 1;

FIG. 3 shows in graphical form the correlation between BBB permeability and volumetric hemispheric expansion;

FIG. 4 is a two-part bar chart showing mast cell blocking and degranulating agents influence edema formation and the degree of BBB damage;

FIG. 5 shows graphically the degree of ischemic brain edema of mast cell deficient rats compared with non-manipulated wild type litter-mates; and

FIG. 6 is a two-part bar chart showing that mast cell inhibition with i.c.v. cromoglycate (Chromo) or mast cell depletion by genetic manipulation reduces postischemic neutrophil infiltration as a component of reperfusion injury.

### DETAILED DESCRIPTION OF THE INVENTION

As noted above, the present invention deals with a therapeutic treatment of the mast cells present in the brain and located typically in perivascular spaces. By preventing degranulation of them, important results can be obtained in the treatment of cerebral ischemia. These features will be examined in more detail below:

As known in the art, mast cells are targets of therapy to treat allergic reactions occurring in asthma or allergic conjunctivitis. Mast cells are also known to be activated and degranulated during mechanical manipulations in the skin as well as trauma to the various parts of the body, and parenchymal organs, but this has not been the basis for any therapeutic methods or modifications of surgical procedures. There are currently no approved medical treatments, which would depend on specific modulation of the mast cell function in the brain, but there have been speculations that they participate in pathophysiologic events of migraine, multiple sclerosis, neuroendocrine reactions, such as psychological stress reactions and Wernicke's encephalopathy, a rare brain disease caused by malnutritional state. In the treatment and various clinical investigations of cerebrovascular disease, such as ischemic and hemorrhagic stroke, brain trauma, brain tumors or increased intracranial pressure, mast cells are not considered as participants of the diseases or targets of therapeutic interventions.

Under conditions of imminent increase of intracranial pressure or edematous cerebral change, there is no obvious physiologic or homeostatic need to allow mast cell degranulation. Such effects, however, must be taken into consideration. Mast cell degranulation liberates numerous vasoactive, proinflammatory and proteolytic substances such as histamine, chemokines, lipases, proteases, kinins, cytokines, arachidonic acid derivatives and nitric oxide. The net effect of liberation of these substances is vasodilation, increase of vascular permeability, inflammatory cell infiltration, edema and tissue expansion (dolor, rubor, tumor). These are unwanted effects in the acute setting of the above hazardous expansive brain diseases. Although there are several other mechanisms by which the brain edema is formed, all elements of edematous brain change need to be eliminated, if possible, at the time when no intracranial reserve spaces are available for further brain expansion, or when such states are imminent or anticipated.

The results of the below examples (in particular Example 1) show that preischemic administration of a well-known mast cell degranulation-blocker, cromoglycate, which prevents mast cell degranulation, into the cerebral ventricles, prevented 39% of the brain edema observed already 3 hours after 60 minutes of middle cerebral artery occlusion in the rat. Furthermore, as also shown in the examples, a mast cell degranulating agent, compound 48/80, administered before reperfusion, aggravated the brain edema by 89%. Furthermore, we have observed that extravasation of plasma proteins from cerebral blood vessels into the extracellular cerebral space in the ischemic brain area was influenced in the same manner as the brain edema by the same treatments.

Therefore, mast cell degranulation seems to participate to a significant degree in ischemic brain edema formation, and the vasogenic component is substantial in the early phase of brain edema development. We have even found that the mast cell degranulation during a 90 minute ischemic period occurs in a subtotal manner, and its effects can be increased even after 85 minutes of ischemia, 8 min prior to the reperfusion by systemically administered further challenges such as compound 48/80.

Therefore, it is believed that a time window exists for prevention of mast cell degranulation and activation even after the onset of cerebral ischemia. This may lead to substantial reduction of the brain edema, inflammation, microvascular stasis and related untoward metabolic tissue events that otherwise would be caused by ischemic or hemorrhagic stroke. Furthermore, unnecessary and potentially harmful mast cell inhibition could be prevented with well-tolerated agents in situations associated with only a risk of cerebral ischemia, and be considered also in mechanical and traumatic manipulations that otherwise activate cerebral or meningeal mast cells.

The above-disclosed effect can be utilized in a number of situations. Thus, potential applications include:

I. Prevention of ischemic brain edema in therapy. These situation are exemplified by surgical procedures associated with a recognizable risk of cerebral ischemia; involving both healthy and diseased subjects. Typical examples are:

1) vascular neurosurgery,

2) heart surgery,

3) carotid artery endarterectomies, and

4) surgery to other major arteries supplying the central nervous tissue

Other application relate to

II. Prevention or alleviation of brain edema in other situations, not involving acute treatment. These are, for example:

1) Surgical procedures associated with physical brain handling of brain and meningeal tissues, that could cause significant mast cell degranulation through mechanical forces,

2) Intracranial expansive diseases such as tumors and epidural/subdural hematomas,

3) Metabolic derangements such as hypoglycemia or hyperglycemia, and

4) The necessity to give adjuvant therapeutic agents to prevent mast cell activation and/or degranulation associated administration of contrast media or other exogenous media for diagnostic or therapeutic purpose into the intravascular, intrathecal or the intracranial space Another area is treatment of ultra-acute and acute phase of stroke and cardiac arrest, such as

1) acute ischemic stroke,

2) acute hemorrhagic stroke,

3) subarachnoid hemorrhage,

4) cerebral venous thrombosis, and

5) global cerebral ischemia associated with cardiac arrest

Disodium cromoglycate is a specific inhibitor of mast cell degranulation. When administered into cerebral ventricles prior to middle cerebral artery occlusion, it prevented almost 40% of the ensuing volumetric expansion of the brain hemisphere in the rat. For human, a critical fraction of brain edema can be prevented, and the exponentially increasing phase of the rise of the intracranial pressure, when the intracranial reserve spaces are exhausted, might in some cases be eliminated. Although the disodium cromoglycate is not effective when given in intravenous infusion, it or similarly acting alternative compounds, such as antihistamines, can be formulated to a compound, which passes the blood-brain barrier and reaches the mast cells positioned often outside the basal membrane in the perivascular spaces. Since extravasation of plasma proteins was also inhibited, the vasogenic portion of the early phase of brain edema in this model is substantial.

Since administration of compound 48/80, a specific mast cell degranulator, after the ischemic period but before the opening of the middle cerebral artery occlusion and reperfusion of its ischemic territory significantly aggravated the volumetric brain by almost 90%, it suggests that a significant fraction of mast cells at that point still remain nondegranulated. While proving that mast cell degranulation can thus be modulated to both decrease and increase brain edema, this also suggests that in already sustained cerebral ischemia, there still exists a therapeutic time window to stabilize non-activated mast cells and prevent brain edema associated with further prolongation of tissue ischemia or reperfusion injury. Further experiments are necessary to delineate the therapeutic time window.

However, mast cell stabilization might be an attractive method to use in the ultra-acute phase of acute strokes, and perhaps could help in some cases to avoid invasive cranial procedures such as hemicraniectomy to control intracranial pressure. Furthermore, similarly acting compounds could be given or applied directly to the intracranial fluid compartment at the beginning of intracranial operations that may provoke unwanted mast cell degranulation. Furthermore, well-tolerated pharmacological formulations based on cromoglycate or other mast cell stabilizing substances might be helpful in other above-listed conditions or diagnostic or therapeutic procedures, where the volumetric enlargement of brain matter or inflammatory or other untoward reactions triggered by mast cell activation are not wanted.

Although many of the triggering factors for mast cell degranulation have been documented, the major trigger in cerebral ischemia remains unknown. One mechanism may relate to complement protein activation, formation of anaphylatoxins C3a and C5a, which are among the most potent activators of mast cells. We have previously demonstrated that ischemic and hemorrhagic brain insults produce activation of the terminal complement pathway, which involves the synthesis of anaphylatoxins. Interestingly, it was demonstrated that even in vitro mixing of ex vivo human plasma and cerebrospinal fluid led to complement activation. Therefore, even temporary and subtle initial breaches of the blood-brain barrier early during ischemia might produce C3a and C5a and trigger degranulation of the mast cells siding the basement membrane and subsequently lead to a more florid blood-brain barrier damage and eventual brain edema. Other mast cell triggering mechanisms could include local production of cytokines such as interleukins of tumor necrosis factor-α.

According to the invention, a mast cell degranulation-blocking or mast cell activation-blocking agent is employed. The agent may have either or both of these activities. The aim is in particular to stabilize the mast cells by, e.g. preventing degranulation of the cells and the release of substances contain in the granulas. Generally speaking, activation of mast cells is to be avoided within the scope of the present invention.

For prevention of cerebral damage, the mast cell degranulation-blocking or mast cell activation-blocking agent should preferably be administered before, preferably at least 5 minutes, in particular at least 10 minutes before the patient is subjected to conditions, which normally are conducive to cerebral ischemia.

The mast cell degranulation-blocking (including mast cell activation-blocking) agent is, according to the invention, preferably selected from the group of 2-carboxylatochromon-5′-yl-2-hydroxypropanederivatives and histamine-1 receptor antagonists. Examples of mast cell degranulation-blocking agents of the first group are bis(acetoxymethyl) cromoglycate, disodium cromoglycate and nedocromil. Further compounds exhibiting selective mast cell degranulation/activation blocking effect include tranilast, and compounds acting primarily on (inhibiting) the c-kit receptor responsible of mast cell maturation and activation, such as imatinibe (e.g. in the form of its mesylate salt).

Examples of the histamine-1 inhibitors are: azatadine, azelastine, burfroline, cetirizine, cyproheptadine, doxantrozole, etodroxizine, forskolin, hydroxyzine, ketotifen, oxatomide, pizotifen, proxicromil, N,N′-substituted piperazines and terfenadine.

Further examples of suitable agents include flavonoids, which inhibit mast cell secretion and proliferation. These are exemplified by quercetin optionally in combination with the proteoglycan chondroitin sulphate. Histamine-2 receptor antagonists, such as cimetidine, optionally combined with e.g. hydroxyzine, along with indolinone derivatives, and IPD-1151T are other examples.

The mast cell degranulation-blocking and/or mast cell activation-blocking agent is administered in a therapeutically efficient amount. Typically, that amount is about 0.05 to 100 milligrams per kilogram body weight of the patient.

The present invention provides for new contrast media, which contain a component preventing or alleviating cerebral ischemia and ischemic edema. Contrast media typically include an iodine-containing component in combination with adjuvants, and have been found to degranulate mast cells. To mention an example, Hexabrix /Guerbet contains sodium and meglumine salts of joxalinic acid, in combination with sodium calcium edetate and water and it has an osmolality of 370 mOsm/kg H₂O. Such a contrast media is, according to the invention, complemented with a suitable amount of a mast cell degranulation-blocking and/or mast cell activation-blocking agent. The amount is generally 0.01 to 100 mg/l.

The compounds employed in the methods of the present invention may be administered by any means that results in the contact of the active agent with the agent's site of action in the body of a patient. The compounds may be administered by any conventional means available for use in conjunction with pharmaceuticals, either as individual therapeutic agents or in a combination of therapeutic agents. For example, they may be administered as the sole active agent in a pharmaceutical composition, or they can be used in combination with other therapeutically active ingredients.

The compounds may be combined with a pharmaceutical carrier selected on the basis of the chosen route of administration and standard pharmaceutical practice as described, for example, in Remington's Pharmaceutical Sciences (Mack Pub. Co., Easton, PA, 1980), the disclosures of which are hereby incorporated herein by reference, in their entireties.

Compounds of the present invention can be administered to a mammalian host in a variety of forms adapted to the chosen route of administration, e.g., orally or parenterally. Parenteral administration in this respect includes administration by the following routes: intravenous, intramuscular, subcutaneous, rectal, intraocular, intrasynovial, transepithelial including transdermal, ophthalmic, sublingual and buccal; topically including ophthalmic, dermal, ocular, rectal, and nasal inhalation via insufflation aerosol. The active compound may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparations should preferably contain at least about 0.1% of active compound. The percentage of the compositions and preparations may, of course, be varied and may conveniently be, for example, from about 2 to about 6% of the weight of the unit. The amount of active compound in such therapeutically useful compositions is preferably such that a suitable dosage will be obtained. Preferred compositions or preparations according to the present invention may be prepared so that an oral dosage unit form contains from about 0.1 to about 1000 mg of active compound, and all combinations and subcombinations of ranges and specific amounts therein.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally or intraperitoneally. Solutions of the active compound as a free base or a pharmacologically acceptable salt can be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compound in the required amount, in the appropriate solvent, with various of the other ingredients enumerated above, as required, followed by filtered sterilization. Generally, dispersions may be prepared by incorporating the sterilized active ingredient into a sterile vehicle that contains the basic dispersion medium and the required other ingredients from those enumerated above. In the case of sterile powders for the preparation of sterile injectable solutions, the preferred methods of preparation may include vacuum drying and the freeze drying technique, which yield a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compound, chosen route of administration and standard pharmaceutical practice.

The dosage of the compounds of the present invention that will be most suitable will vary with the form of administration, the particular compound chosen and the physiological characteristics of the particular patient under treatment.

In particular, adminstration can be carried out parenterally, for example by i.v., i.c.v. (intracerebroventricularly) and i.m. administration. Parenteral compositions usually contain a buffering agent and, optionally, a stabilizing agent.

When necessary, in order to promote penetration of the blood-brain-barrier, the active compounds can be administered by using various now strategies for gaining drug access to the brain. These include the transcellular lipophilic pathway, which allows small, lipophilic compounds to cross the blood-brain barrier. A second pathway is “receptor-mediated endocytosis. Further, as known in the art, some experimental work has shown that a monoclonal antibody for the transferrin receptor, coupled with brain-derived neurotrophin factor, which is neuroprotective but cannot cross the barrier itself, can both cross the barrier and exert neuroprotective effects. Endothelial cells of the blood-brain barrier also express a number of transport proteins, including transporters for glucose, amino acids, nucleosides, and other compounds. Thus, to focus on the latter strategy, the compounds can be designed such that they gain access to the brain by going through these transport processes. It is, however, also possible to block these processes, in that way bolstering brain levels of endogenous permeant.

For the sake of completeness, it should be pointed out that the compounds employed in the uses and methods of the present invention may exist in prodrug form. As used herein, the term “prodrug” is intended to include any covalently bonded carriers which release the active parent drug or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.), the compounds employed in the present methods may, if desired, be delivered in prodrug form. Thus, the present invention contemplates methods of delivering prodrugs. Prodrugs of the compounds employed in the present invention may be prepared by modifying functional groups present in the compound in such a way that the modifications are cleaved, either in routine manipulation or in vivo, to the parent compound.

Accordingly, prodrugs include, for example, compounds described herein in which a hydroxy, thiol, amino, or carboxy group is bonded to any group that, when the prodrug is administered to a mammalian subject, cleaves to form a free hydroxyl, thiol, free amino, or carboxylic acid, respectively. Examples include, but are not limited to, acetoxyalkyls, acetate, formate and benzoate derivatives of alcohol, thiol, and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

Next, the invention will be illustrated with the aid of two examples:

### EXAMPLE 1

Methods

The suture filament model was used to induce focal cerebral ischemia for 60 min in rats. Reperfusion was allowed for 3 h, at which point the rats were killed, cardioperfused and their brains were dissected into coronal sections. Evans Blue-albumin (2%, 0.3 ml/100 g) a fluorescent dye, was injected i.v. 20 min before termination to monitor BBB (blood brain barrier) permeability. TTC (2,3,5-triphenyltetrazolium chloride, 2%) staining was used to quantitate the infarct volumes. The volumetric expansion of the ischemic hemisphere was quantitated with computerized planimetry. Rats were assigned in three pharmacological treatments: mast cell stabilizer disodium cromoglycate 750 ug in 10 ul i.c.v. (does not easily cross BBB) (n=14) or control (10 ul saline i.c.v.) (n=13) 5 min prior to ischemia, and a mast cell degranulation agent, compound 48/80 (n=11) administered (0.025 mg i.v.) 3 min prior to reperfusion. The Evans Blue extravasation was analysed using fluorescence microscopy and computerized image analysis-based quantitation of the fluorescent pixels in five random regions of interest.

Results

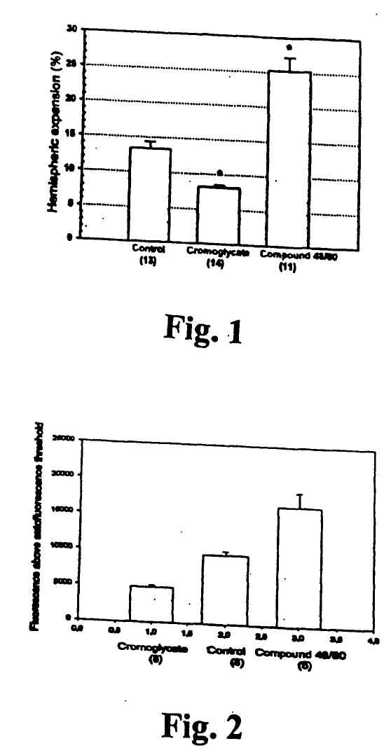

The volumetric hemispheric expansion caused by ischemia was highly significantly influenced by pharmacologic modulation of mast cell degranulation (Kruskal-Wallis ANOVA p<0.001, FIG. 1), as well as the mean number of fluorescent pixels indicating rates of extravasation (p<0.001, FIG. 2). Corrected infarct volumes were not influenced by the treatments: control 189±31, cromoglycate 252±41, compound 48/80 193±24 mm³, p=0.33.

The results confirm the observations discussed above: inhibition of mast cell degranulation by intraventricular administration of cromoglycate led to a 39% reduction of the acute-phase ischemic edema.

### EXAMPLE 2

In this example it is shown that pharmacological prevention of degranulation by intracerebroventricular (i.c.v.) infusion of disodium cromoglycate, a selective MC stabilator, strongly inhibits early ischemic brain edema and BBB permeability increase, but induces MC degranulation with the classic MC secretagogue, Thus, compound 48/80 used in the example, significantly aggravates both of these phenomena. MC-deficient WsRc^{Ws/Ws} rats carrying a defective gene for c-kit (ligand for stem cell factor [SCF] required for MC differentiation were subjected to transient focal cerebral ischemia, and it was demonstrated that early ischemic BBB damage and brain edema are influenced by MC degranulation. Finally, the early neutrophil response, a component of early inflammation and reperfusion injury, was shown to be dependent on the presence of MC, and decreased by pharmacological MC stabilation. The mortality and neurological deterioration after hemorrhagic brain injury was shown to be reduced by MC inhibition.

Methods

We used adult male Wistar rats (Harlan Nederland), and MC-deficient, WsRc^{Ws/Ws} rats (Japan SLC, Inc., Japan), 290 to 340 grams. We anesthetized the rats by an intraperitoneal injection of ketamin hydrochloride (50 mg/kg, Ketalar) and a subcutaneous injection of medetomidin hydrochloride (0.5 mg/kg, Domitor). A PE-50 polyethylene tube was placed into the left femoral artery for continuous monitoring of blood pressure and another into the left femoral vein for drug and/or vehicle infusions. Rectal temperature was monitored and maintained the temperature at 37° C. during the operation with a heating blanket and a thermo-regulated heating lamp.

Middle Cerebral Artery Occlusion

We used the suture MCA occlusion model with reperfusion, described elsewhere. Briefly, we exposed the right common carotid artery (CCA) and the right external carotid artery (ECA) through a ventral midline neck incision. We ligated the proximal CCA and the origin of the ECA. We inserted a 4-0 nylon mono-filament suture (Ethilon Nylon Suture, ETHICON) with its tip rounded by heating near a flame and then coated with silicone into the right CCA via an arteriectomy. Reperfusion was accomplished by withdrawing the suture occluder 60 minutes after MCAO, whereafter the animals were allowed to recover. Four hours after MCAO, the rats were killed by injecting intraperitoneally 120 mg of pentobarbital natrium (Mebumat). Cardiac perfusion was performed with the heart still beating but respiration ceased by infusing 250 mL of ice cold 0.9% saline at an inflow pressure of 100 mmHg into the arterial vascular system.

Brain Hemorrhage Protocol

In anesthesized rats, 2 craniotomies were performed for the introduction of drug infusion and blood. We injected 50 microliters of autologous arterial blood via stereotactically a Hamilton needle tip placed to the caudate putamen (6 mm from skull surface), a common location of spontaneous cerebral hemorrhages.

Animals were allowed to recover and 24 h later their level of neurological deterioration was rated on a 6-point scale: 5=dead, 4=stuporous, moribund, 3=hemiparetic with balance disturbance, 2=hemiparesis, 1=paresis of contralateral paw, 0=normal. The mortality was also recorded by 24 hours. Furthermore, we will analyze the imaging results of cerebral magnetric resonance (MS) images performed serially with 4.7 Tesla (Bruker) MR scanner designed for small animal studies. These analyzes (T2*, T2 and diffusion-weighted images) will show the reduction of cerebral edema in this animal series.

Tissue Handling

After cardiac perfusion, the brains were quickly removed and dissected coronally into six 2-mm slices using a standard brain-cutting matrix. The 3^{rd} slice was cut into two 1-mm sections. The rostral section was embedded in Tissue-Tek, snap-frozen in liquid nitrogen and kept thereafter at −80° C. until 15 μm sections were cut for fluorescence microscopy. The caudal middle section, together with the other five 2-mm brain slices, were incubated for 15 minutes in TTC at 37° C., and immersion-fixated subsequently in 10% formaldehyde.

Histopathological Examinations

5 μm tissue sections were cut and stained with Toluidine blue, a standard metachromatic histopathological technique to detect the heparin-containing granules of MC, and the chloroacetate Leder staining to detect also neutrophils. Light microscopy was performed by an experienced hematopathologist with Olympus BH-2 and photographed with Nikon microscope connected with a Nikon Coolpix digital camera. The densities of intravascular and emigrated neutrophils were counted without prior knowledge of animal grouping in systematically placed target areas, the temporoparietal infarction core, the parasagittal infarct penumbra and the deep thalamic and basal ganglia. In each area, 60 to 100 microscopic fields of 0.1 mm² were counted and averaged for each location.

Pharmacological Protocols and Animal Grouping

For pharmacological MC modulations, we used disodium cromoglycate as a well-documented and highly specific MC stabilizer and Compound 48/80 as a standard MC degranulating secretagogue. The experimental protocol was conducted in a blinded and randomized manner. In the first part of the study, we divided animals into three treatment protocols. Group A (n=14) received cromoglycate (750 μg) administered 5 minutes before MCA occlusion i.c.v., since it does not easily cross the BBB, dissolved in saline to a final volume of 10 μl, and vehicle (0.1 ml of saline) 3 minutes before reperfusion. Group B (n=11) received vehicle (10 μl saline i.c.v.) 5 minutes before MCA occlusion, and Compound 48-80 (0.25 mg/mL) i.v., reaching the brain when given i.v. 15, dissolved in saline to a final volume of 0.1 ml at 3 minutes before reperfusion. Group C (n=13) received vehicle (10 μl saline i.c.v.) 5 minutes before MCA occlusion and vehicle (0.1 ml of saline) 3 minutes before reperfusion. In the second part of the study we performed MCAO in mast-cell deficient rats WsRc^{Ws/Ws} (group D, n=10) and their non-deficient litter-mates (group B, n=8).

In the surgically induced cerebral hemorrhage experiments, animals were grouped to control (saline) i.c.v. (N=10) and i.c.v cromoglycate N=10).

BBB Damage Evans blue albumin (EBA) (Sigma, 20 mg/mL dissolved in 1% albumin), a fluorescent dye, was used to visualize BBB damage to the molecular size of serum albumin. Animals received 2% solution of EBA into the femoral vein (0.3 mL/10 mg) 20 minutes before cardiac perfusion.

In the pharmacologically modulated groups, the brain slices were mounted and examined for the distribution of characteristic red fluorescence of the EBA tracer in the brain parenchyma using epifluorescent microscope Axioplan 2 (Carl Zeiss Vision GmbH) and a dedicated Evans blue fluorescence filter (Chroma Technology Corp.). Fluorescent images of brain sections were captured with AxioCAM HR digital camera (Carl Zeiss Vision GmbH) for evaluation of BBB damage. The reliability of digital imaging fluorescence microscopy in the measurement of tracer concentration in sectioned tissue was previously reported. Briefly, we first calibrated the image analyzing software for the auto-fluorescence level represented by the amount of fluorescence pixels in the healthy hemisphere, using this as a threshold level. We then calculated the total amount of EBA-fluorescent pixels in the infarcted hemisphere. Finally, we calculated the difference in fluorescence intensities between the treatment groups. In the WsRc^{Ws/Ws} rat series a simpler method was used, as we calculated the difference in the absolute amount of fluorescence pixels between the infarcted and healthy hemisphere using fluorescence scanner Typhoon 9400 (Amersham Biosciences UK Ltd).

Calculation of Infarct and Edema Volume

For each animal, all 6 TIC-stained brain slices were photographed, mounted on a scale-paper, using a digital camera (Sony, Mavica, Japan) attached to fixed tripod. Infarction and corrected infarction volumes were calculated as we have described elsewhere⁴¹. We measured the percentage of hemispheric expansion, which indicates the volumetric increase of the ischemic hemisphere in comparison to the healthy one (% of hemispheric expansion=(right hemisphere's volume/left hemisphere's volume)−1 * 100).

Image Analyses

We used NIH Image Analyses Software (NIH, USA), Adobe Photoshop 6.0 (Adobe Systems Incorporated, USA), KS 300 Image Analysis System (Carl-Zeiss Vision GmbH, Germany) and Amersham (both) for image analyses in this study.

Results

Infarct Volumes

Corrected infarct volumes measured based on 2,3,5-triphenyltetrazolium chloride (TTC) stained areas were not significantly influenced by the treatment assignments.

Brain Edema

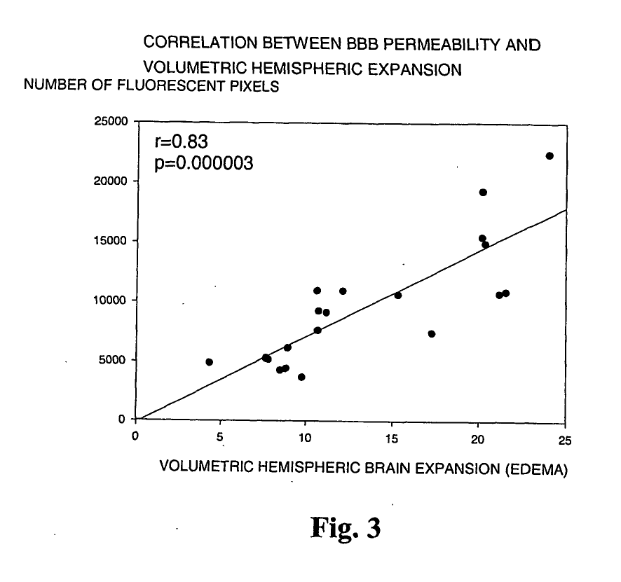

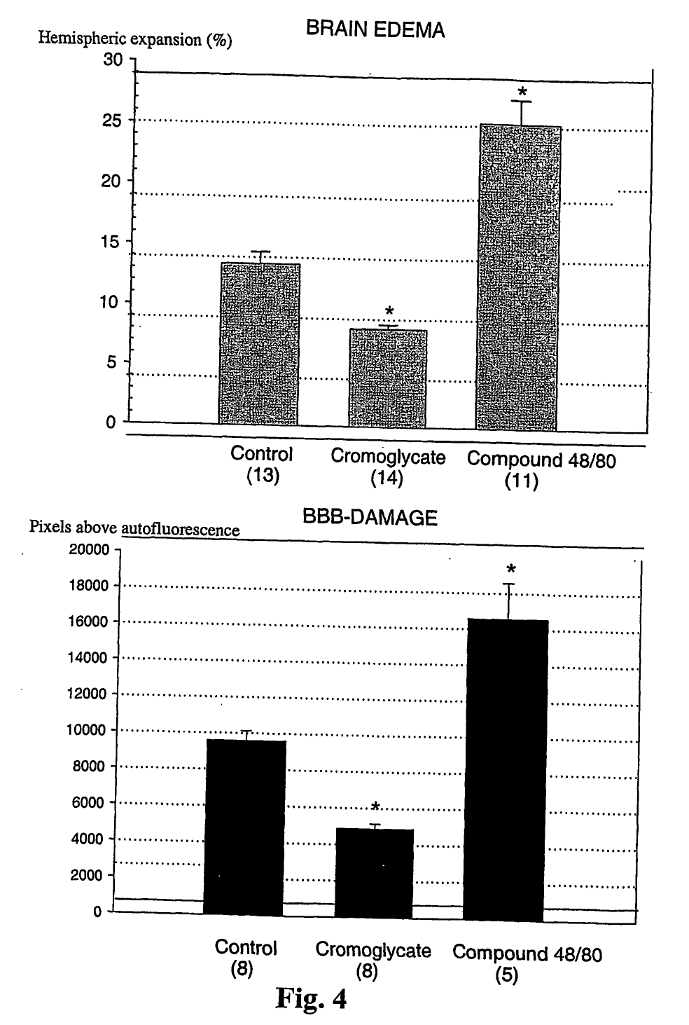

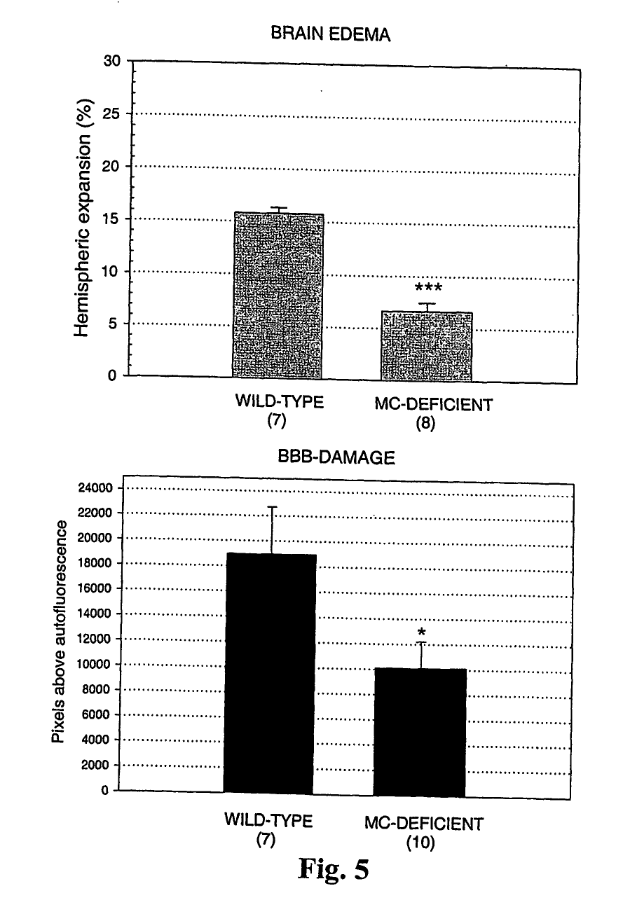

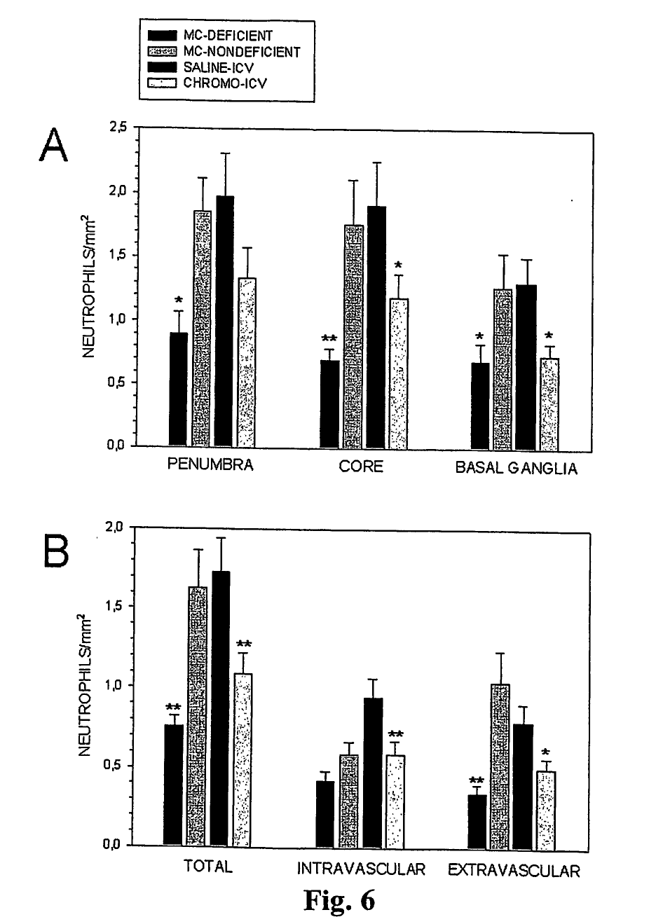

The volumetric hemispheric expansion caused by ischemia was highly significantly influenced by pharmacological modulation of MC degranulation state with cromoglycate (FIG. 2A) as well as between the WsRc^{Ws/Ws} rats and their controls (P<0.001, FIG. 3A). The percentage of the volumetric expansion was 8.13±1.37 in the cromoglycate treated group; 25.23±6.67 in the Compound 48/80 treated group; and 13.37±3.57 in the control group (FIG. 4). In the WsRc^{Ws/Ws} rats the volumetric expansion was 6.67±1.25, and 15.78±1.45 in their non-manipulated controls.

BBB Integrity

Fluorescence microscopic visualization of Evans Blue albumin (EBA) in the pharmacologically modulated experimental groups, as well as fluorescence scanning in the WsRc^{Ws/Ws} rats suggested that the rates of extravasation were differentially regulated. While the EBA-fluorescence in the cromoglycate-treated rats was punctuate and seemed to focus on small vessels or their endothelia, the compound 48/80 treated ones had more diffuse fluorescence forming even confluent areas. The number of fluorescence pixels indicating rates of extravasation were highly significantly decreased by sodium cromoglycate, when compared to the control (FIG. 2B, P<0.001) respectively, and increased by Compound 48/80, when compared to the control (P<0.001), 16578±2008 and 9604±524 respectively (FIG. 2B). In line, the difference in the absolute amount of fluorescence pixels between the infarcted and healthy hemisphere in the WsRc^{Ws/Ws} rats was highly significantly reduced when compared to their control (P<0.001), 10140±6557 and 18972±9867 respectively (FIG. 3B).

Neutrophil Response

Already at this early postischemic time point, neutrophil density was increased up to 2.5-fold in the infarcted hemisphere in comparison to the non-infarcted hemisphere. Lowest counts of neutrophils were found in the genetically MC-deficient rats, which had 47% of the neutrophil count found in the infarcted hemisphere of their unaltered litter-mates (p=0.002, FIG. 4). Sodium cromoglycate significantly decreased the density of neutrophils in the infarcted hemisphere by 37% (p-0.006). Interestingly, sodium cromoglycate decreased not only the number of emigrated neutrophils but also those detected still within the intravascular space (FIG. 4B). The difference was most pronounced in the infarct core and basal ganglia.

Cerebral Hemorrhage Outcome

The mortality of the cerebral hemorrhage induced by surgical procedures in i.c.v. saline-treated group was 40% while it was 0% in the i.c.v. cromoglycate-treated group. In the six-point neurological score scale, the cromoglycate-treated fared better (0.89 on the average) than the saline-treated (3.2 on the average), which differrence was statistically significance (p<0.01). The results of the edema changes measured with MR imaging as well as in histological section at 24 hours after the hemorrhage will show the efficacy of cromoglycate in reducing edema and secondary hemorrhage caused by these surgical manipulations and hemorrhagic brain injury.

To clarify the duration of the therapeutic time window we have demonstrated, we perform new experiments where cromoglycate is administered to groups of rats at variable periods, 30 min, 60 min, 120 min and 240 min after the onset of ischemia induced by MCAO, following the methodology described above. In these experiments, a i.c.v. drug delivery system is devised to allow administration in awake animals. These studies demonstrate the value of this therapeutic approach in relevance to human patients who have had a variable symptom duration after the onset of stroke symptoms.

To make drug delivery route more practical especially in the clinical setting, rat MCAO model can be used as described in detail in this application by replacing i.c.v. drug administration as the pretreatment before thrombolysis with intravenous (i.v.) drug administration of an alternative mast cell degranulation-blocking and/or mast cell activation-blocking agent, which possess the capacity to penetrate through blood-brain barrier more effectively than sodium cromoglycate.

Furthermore, experiments can be performed that use radiological contrast media in rats subjected to subthreshold ischemia severity, 30 minutes of MCAO producing focal ischemic insult that is thereafter imaged with MRI to determine the cerebral lesion size. We compare groups that receive sodium cromoglycate or saline i.c.v. followed by an intravenous radiographic contrast agents (such as diatrizoate, ioxaglate, iotrolan) at 5 minutes after reperfusion. These experiments will indicate the potential edematous or blood extravasation promoting effects of MC activating contrast agents. The usefulness of MC-inhibitors in preventing these deleterious effects in the setting of imminent cerebral infarction is demonstrated by shrinking of edematous or diffusion-weighted lesions in serial MRI illustrated lesion. At 24 hours, the size of cerebral swelling and the area of neuronal damage on histological sections can be determined.

Discussion

MCs are the well-known cellular mediator of immediate hypersensitivity and local phlogistic reaction to numerous mechanical, toxic and allergic stimuli. The present invention demonstres that cerebral MC mediate early ischemic edema, BBB-failure and inflammatory cell reaction. Pharmacological inhibition of MC degranulation by sodium cromoglycate led to 39% reduction of the acute ischemic edema caused by transient MCAO, whereas the MC degranulating agent Compound 48/80 increased the edema formation by 89% in comparison to the control group. To prove the specificity of the MC-inhibiting treatments, genetically altered WsRc^{Ws/Ws}-rats, which are devoid of MC, exhibited 58% reduction of the acute ischemic edema, when compared to their non-altered litter-mates. Thus, cerebral MC and their secretory granule content contribute to the acute BBB permeability change after focal transient cerebral ischemia, and this can be pharmacologically modulated. This new art is interesting in a number of clinical situations, where anticipated cerebral ischemia may be complicated by hazardous brain edema and florid extravasation. Furthermore, this new art describes a method to prevent mortality and neurological deterioration after surgical procedures and induced brain hemorrhage. Ischemic BBB damage has long been held to be biphasic, the first phase of which beginning already within minutes. The cellular or molecular mediators of especially the first phase have not been characterized. The integrity of BBB is largely determined by the basal lamina, the main three constituents of which are matrix proteins laminin, fibronectin and collagen type IV. There are reports suggesting that the integrin-labeled basal lamina loses its integrity very early after the onset of cerebral ischemia. Cerebral MC are known to contain chymase in secretory granules. Chymase is an efficient protease, the substrate of which includes fibronectin and it can also activate procollagenases (matrix metallo-proteinases), even in the presence of tissue inhibitor of metalloproteinase (TIM)-1. MC also directly release gelatinases A (MMP-2) and B (MMP-9), which are well-known to degrade collagen type IV of the basement membrane. Although more ultrastructural and immunocytochemical confirmatory studies are required, we raise the possibility that, at least in scattered densely ischemic foci, MC-derived chymase and collagenase activity and may participate in this early phase of molecular degradation of basal lamina after ischemia. Thus, it can be concluded that the effects of MC-blocking on BBB permeability permits a pharmacologic approach to prevent imminent ischemic brain edema, which is a devastating complication of acute stroke and the most frequent mechanism of early death in stroke. The inflammatory cell response is also be inhibited through interventions blocking MC activation. Furthermore, MC-inhibition reduced mortality and neurological deterioration after cranial surgery and intracranial hemorrhage. This scenario and the present data make specific MC-inhibitors an interesting art in CNS conditions where increased BBB permeability and inflammation plays a pathological role.

1-15. (canceled)

16. A method of treating a patient suffering from cerebral ischemia, comprising the step of administering to the patient a pharmaceutically effective amount of a mast cell activation- or degranulation-blocking agent.

17. The method according to claim 16, wherein the patient is suffering from acute ischemic stroke, acute hemorrhagic stroke, subarachnoid hemorrhage, cerebral venous thrombosis or global cerebral ischemia associated with cardiac arrest.

18. The method according to claim 16, wherein the patient is suffering from an intracranial expansive disease, such as tumors or epidural/subdural hematomas, or metabolic derangements, such as hypoglycemia or hyperglycemia

19. The method according to claim 16, wherein the patient is suffering from ischemic brain edema.

20. The method according to claim 16, wherein the mast cell activation- or degranulation-blocking agent is selected from the group consisting of 2-carboxylatochromon-5′-yl -2-hydroxypropane derivatives, histamine-1 receptor antagonists, flavonoids and histamine-2 receptor antagonists.

21. The method according to claim 20, wherein the mast cell activation- or degranulation-blocking agent is selected from the group consisting of bis(acetoxymethyl) cromoglycate, disodium cromoglycate, nedocromil, tranilast, c-kit receptor inhibitors, chymase inhibitors and precollagenase activator inhibitors.

22. The method according to claim 20, wherein the mast cell activation or degranulation-blocking agent is selected from the group consisting of azatadien, azelastine, cetirizine, cyproheptadien, doxantrozole, etodroxizine, forskolin, hydroxyzine, ketotifen, oxatomide and terfenadine.

23. The method according to claim 21, wherein the mast cell activation- or degranulation-blocking agent is administered in an amount of about 0.05 to 100 milligrams per kilogram body weight of the patient.

24. The method according to claim 16, wherein the agent is present in a pharmaceutical composition formulated for parenteral administration.

25. The method according to claim 24, wherein the pharmaceutical composition comprises a buffering agent and a stabilizing agent.

26. A method of preventing or reducing mast cell degranulation in a patient subjected to a condition conducive to cerebral ischemia, comprising administering to said patient a therapeutically effective amount of a of a mast cell activation- or degranulation-blocking agent.

27. The method according to claim 26, wherein the patient is subjected to a surgical procedure associated with a recognizable risk of cerebral ischemia, the agent being administered to the patient before the initiation of the surgical procedure.

28. The method according to claim 27, wherein the patient is subjected to vascular neurosurgery, heart surgery, carotid artery endarterectomies or surgery to other major arteries supplying the central nervous tissue.

29. The method according to claim 28, wherein the patient is subjected to a surgical procedure associated with physical brain handling of brain and meningeal tissues, or radiation, that could cause significant mast cell degranulation through mechanical forces.

30. The method according to claim 26, wherein the patient is subjected to a treatment involving the administration of contrast media or other exogenous media for diagnostic or therapeutic purpose into the intravascular, intrathecal or the intracranial space.

31. The method according to claim 26, wherein the mast cell activation- or degranulation-blocking agent is selected from the group consisting of 2-carboxylatochromon-5′-yl -2-hydroxypropane-derivatives, histamine-1 receptor antagonists, flavonoids, and histamine-2 receptor antagonists.

32. The method according to claim 31, wherein the mast cell activation- or degranulation-blocking agent is selected from the group consisting of bis(acetoxymethyl) cromoglycate, disodium cromoglycate, nedocromil, tranilast c-kit receptor inhibitors, chymase inhibitors and precollagenase activator inhibitors

33. The method according to claim 31, wherein the mast cell activation- or degranulation-blocking agent is selected from the group consisting of azatadine, azelastine, cetirizine, cyproheptadine, doxantrozole, etodroxizine, forskolin, hydroxyzine, ketotifen, oxatomide and terfenadine.

34. The method according to claim 31, wherein the agent is present in a pharmaceutical composition formulated for parenteral administration.

35. A composition of contrast medium or a similar exogenous medium useful in diagnostic or therapeutic applications for introduction into the intravascular, intrathecal or the intracranial space, comprising a mast cell degranulation-blocking and/or mast cell activation-blocking agent.

36. The composition according to claim 35, further comprising an iodine-containing active component in combination with a pharmacologically acceptable adjuvants and containing said mast cell degranulation-blocking and/or mast cell activation-blocking agent in a therapeutically effective amount to prevent or reduce any cerebral ischemia caused by the active component.

37. The composition according to claim 36, wherein the amount of the mast cell degranulation-blocking and/or mast cell activation-blocking agent is 0.01 to 100 mg/l.

38. The composition according to claim 35, wherein the mast cell degranulation-blocking and/or mast cell activation-blocking agent is selected from the group consisting of bis(acetoxymethyl) cromoglycate, disodium cromoglycate, nedocromil, tranilast c-kit receptor inhibitors, chymase inhibitors and precollagenase activator inhibitors.

39. The composition according to claim 35, wherein the mast cell activation- or degranulation-blocking agent is selected from the group consisting of azatadine, azelastine, cetirizine, cyproheptadine, doxantrozole, etodroxizine, forskolin, hydroxyzine, ketotifen, oxatomide and terfenadine.
